(19) United States
(12) Patent Application Publication
(10) Pub. No.: US 2019/0351912 A1
(43) Pub. Date: Nov. 21, 2019

(54) SYSTEM FOR DETERMINING DRIVER'S EMOTION IN VEHICLE AND CONTROL METHOD THEREOF

(71) Applicants: HYUNDAI MOTOR COMPANY, Seoul (KR); KIA MOTORS CORPORATION, Seoul (KR)

(72) Inventors: Seunghyun WOO, Seoul (KR); Gi Beom HONG, Seoul (KR); Daeyun AN, Anyang-Si (KR)

(21) Appl. No.: 16/191,040

(22) Filed: Nov. 14, 2018

(30) Foreign Application Priority Data
May 18, 2018 (KR) 10-2018-0056829

(51) Int. Cl. B60W 50/00; G06F 3/16; B60H 1/00; B60W 40/08; A61B 5/16; A61B 5/00; A61M 21/00

(52) U.S. Cl. CPC B60W 50/0098; G06F 3/165; B60H 1/00964; B60W 40/08; A61B 5/165; A61B 5/0245; A61B 5/7264; A61M 21/00; B60W 2540/22; B60W 2710/30; A61B 5/0077; A61B 5/6893

(57) ABSTRACT

a vehicle includes: a sensor configured to sense a condition of a user using at least one sensor, a storage configured to store information on a relationship between the sensor and an emotional factor and feedback information for the user with regard to the emotional factor, and a controller configured to acquire information on the current emotional condition of the user based on values measured by the sensor and control a feedback device provided in the vehicle so that the current emotional condition of the user reaches a target emotion.

### CROSS-REFERENCE TO RELATED APPLICATION(S)

This application is based on and claims priority under 35 U.S.C. § 119 to Korean Patent Application No. 10-2018-0056829, filed on May 18, 2018 in the Korean Intellectual Property Office, the disclosure of which is incorporated by reference herein in its entirety.

### TECHNICAL FIELD

The present disclosure relates to a vehicle and a control method thereof, and more particularly, to a vehicle and a control method thereof capable of providing appropriate feedback to a driver based on an emotional state of the driver.

### BACKGROUND

In modern society, vehicles are one of the most common means of transportation and the number of people using them is continuously increasing. With the development of vehicle technologies, there have been many changes in life, e.g., it is easier to travel a long distance, etc.

In recent years, technologies have been developed to determine a driver's emotion in consideration of the driver's mood and to increase the driver's convenience according to the emotion. For example, technologies using biometrics to determine the driver's emotion have been developed.

Biometrics, which recognizes a part of a person's body and performs emotion determination, includes voice recognition, face recognition, hand gesture recognition, or heartbeat recognition. Since biometrics uses a part of the body that changes according to the mood of the person, accuracy of the biometrics will increase if an emotion of the person is determined. Accordingly, many studies on determination of emotion are being conducted.

### SUMMARY

It is an aspect of the present disclosure to provide a vehicle and a control method thereof capable of determining a current emotional state of a driver and providing appropriate feedback to the driver based on the current emotional state.

Additional aspects of the present disclosure will be set forth in part in the description which follows and, in part, will be obvious from the description, or may be learned by practice of the disclosure.

In accordance with an aspect of the present disclosure, a vehicle may include: a sensor configured to sense a condition of a user using at least one sensor, a storage configured to store information on a relationship between the sensor and an emotional factor and feedback information for the user with regard to the emotional factor, and a controller configured to acquire information on a current emotional condition of the user based on values measured by the sensor and control a feedback device provided in the vehicle so that the current emotional condition of the user reaches a target emotion.

The controller may be configured to classify the current emotional condition of the user and the target emotion according to a preset reference, and then control the feedback device based on the classification result.

The controller may be configured to, when the current emotional condition of the user corresponds to a first emotion, control the feedback device so that the emotional condition of the user is maintained at the first emotion.

The controller may be configured to, when the current emotional condition of the user corresponds to a second emotion, control the feedback device so that the emotional condition of the user reaches a first emotion.

The controller may be configured to extract emotional factors affecting the current emotional condition of the user, and then control the feedback device in a way of boosting or reducing the extracted emotional factors.

The controller may be configured to, when the emotional factors belong to a first group, control the feedback device in a way of boosting the emotional factors.

The controller may be configured to, when the emotional factors belong to a second group, control the feedback device in a way of reducing the emotional factors.

The feedback device may include at least one of a multimedia device, an air conditioner, a display, a speaker, and a ventilator provided in the vehicle.

The controller may be configured to control at least one of volume, genre, equalizer, tone, and acoustic wave band of music played in the vehicle.

The vehicle may further include an input device configured to receive information on the target emotion from the user.

In accordance with another aspect of the present disclosure, a control method of a vehicle may include: sensing a condition of a user using at least one sensor, receiving information a relationship between the sensor and an emotional factor and feedback information for the user with regard to the emotional factor, and acquiring information on the current emotional condition of the user based on values measured by the sensor and controlling a feedback device provided in the vehicle so that the current emotional condition of the user reaches a target emotion.

The controlling of the feedback device may include classifying the current emotional condition of the user and the target emotion according to a preset reference, and controlling the feedback device based on the classification result.

The controlling of the feedback device may include, when the current emotional condition of the user corresponds to a first emotion, controlling the feedback device so that the emotional condition of the user is maintained at the first emotion.

The controlling of the feedback device may include, when the current emotional condition of the user corresponds to a second emotion, controlling the feedback device so that the emotional condition of the user reaches a first emotion.

The controlling of the feedback device may include extracting emotional factors affecting the current emotional condition of the user, and controlling the feedback device in a way of boosting or reducing the extracted emotional factors.

The controlling of the feedback device may include, when the emotional factors belong to a first group, controlling the feedback device in a way of boosting the emotional factors.

The controlling of the feedback device may include, when the emotional factors belong to a second group, controlling the feedback device in a way of reducing the emotional factors.

The feedback device may include at least one of a multimedia device, an air conditioner, a display, a speaker, and a ventilator provided in the vehicle.

The controlling of the feedback device may include controlling at least one of volume, genre, equalizer, tone, and acoustic wave band of music played in the vehicle.

The control method may further include receiving information on the target emotion from the user.

### BRIEF DESCRIPTION OF THE DRAWINGS

FIG. 1 is a view illustrating the interior of a vehicle according to an embodiment of the present disclosure;

FIG. 2 is a block diagram illustrating some components of an emotion mapping apparatus according to an embodiment of the present disclosure;

FIG. 3 is a flowchart illustrating a control method of an emotion mapping apparatus according to an embodiment of the present disclosure;

FIG. 4 is a table illustrating information on correlations between sensors and emotional factors;

FIGS. 5A and 5B are tables illustrating emotional factors extracted as having correlations with sensors that exceed a preset reference;

FIG. 6 is a view illustrating an emotion map generated according to an embodiment of the present disclosure;

FIG. 7 is a block diagram illustrating some components of a vehicle according to an embodiment of the present disclosure;

FIG. 8 is a flowchart illustrating a control method of a vehicle according to an embodiment of the present disclosure;

FIG. 9 is a table showing correlation information between emotional factors and feedback elements;

FIGS. 10A and 10B are tables illustrating emotional factors extracted as having correlations with feedback elements that exceed a preset reference; and

FIGS. 11 to 13 are diagrams illustrating a method of making a user's emotional state reach a target emotion.

### DETAILED DESCRIPTION

Like reference numerals refer to like elements throughout the specification. This specification does not describe all the elements of the embodiments, and duplicative contents between general contents or embodiments in the technical field of the present disclosure will be omitted. The terms ‘part,’ ‘module,’ ‘member,’ and ‘block’ used in this specification may be embodied as software or hardware, and it is also possible for a plurality of ‘parts,’ ‘modules,’ ‘members,’ and ‘blocks’ to be embodied as one component, or one ‘part,’ ‘module,’ ‘member,’ and ‘block’ to include a plurality of components according to the embodiments.

Throughout the specification, when a part is referred to as being “connected” to another part, it includes not only a direct connection but also an indirect connection, and the indirect connection includes connecting through a wireless network.

When it is described that a part “includes” an element, it means that the element may further include other elements, not excluding the other elements unless specifically stated otherwise.

Throughout the specification, when it is described that a member is located “on” another member, this includes not only when a member is in contact with another member, but also when there is an intervening member between the two members.

The terms ‘first,’ ‘second,’ etc., are used to distinguish one element from another element, and the elements are not limited by the above-mentioned terms.

The singular forms “a,” “an,” and “the” include plural referents unless the context clearly dictates otherwise.

In each step, an identification sign is used for the convenience of explanation, and the identification sign does not describe the order of each step, and each step may be performed differently from the stated order unless clearly specified in the context.

Hereinafter, the working principle and embodiments of the present disclosure will be described with reference to the accompanying drawings.

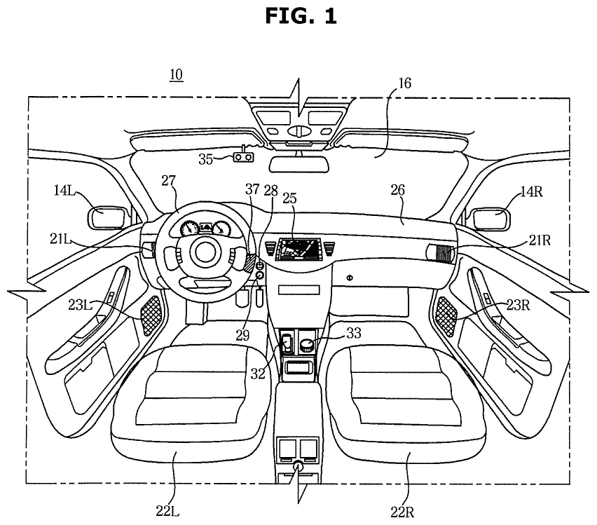

FIG. 1 is a view illustrating an interior of a vehicle provided with an emotion mapping apparatus according to an exemplary embodiment of the present disclosure.

Referring to FIG. 1, in the central area of a dashboard 26, a navigation device 25 for displaying various videos or images in addition to driving information of a vehicle 100 may be provided.

The navigation device 25 may perform a function of providing a user with a route to a destination or providing map information about a specific location. Devices that perform this function are generally called navigation devices or GPS navigation devices, but may also have many different names, which are commonly called by those of ordinary skill in the art.

The navigation device 25 may include a display for displaying various videos and images including the driving information of a vehicle.

A center input 33 of a jog shuttle type may be provided between a driver's seat 22L and a passenger's seat 22R. The user may input a control command by turning, pressing, or pushing the center input 33 upward, downward, to the left or right.

The vehicle 100 may be provided with speakers 23L and 23R capable of outputting sound.

The speakers 23L and 23R may output sounds necessary for performing an audio function, a video function, a navigation function, and other additional functions.

In FIG. 1, the speakers 23L and 23R are provided in the driver's seat 22L and the passenger's seat 22R, respectively. However, the positions of the speakers 23L and 23R are not limited thereto and may be anywhere in the vehicle 100.

A steering wheel 27 is provided on the dashboard 26 on the driver's seat 22L side, and a key groove 28 for inserting a remote control device (not shown), for example, a Free On Board (FOB) key, may be formed in an area adjacent to the steering wheel 27. When the remote control device capable of turning on/off the ignition of the vehicle 100 is inserted into the key groove 28 or authentication between the remote control device and the vehicle 100 is completed via a wireless communication network, an external terminal (not shown) may be connected to the vehicle 100.

Further, the dashboard 26 may be provided with a start button 29 for controlling on/off of the ignition of the vehicle 100. If the remote control device capable of controlling the vehicle 100 is inserted into the key groove 28 or authentication between the external terminal and the vehicle 100 is successfully performed through the wireless communication network, the ignition of the engine 100 may be turned on when the start button 29 is pushed by the user.

The vehicle 100 may be provided with an air conditioner to perform both heating and cooling, and may control the temperature inside the vehicle 100 by discharging the heated or cooled air through air vents 21L and 21R.

In FIG. 1, the air vents 21L and 21R are provided in front of the driver's seat 22L and the passenger's seat 22R, respectively. However, the position of the air vents 21L and 21R is not limited thereto and may be anywhere in the vehicle 100.

Referring to FIG. 1, a variety of biometric devices may be provided in the vehicle 10 to determine an emotion of the driver. The biometric devices may include, but not exclusively, a camera 35 for recognizing the face or hand motion of the driver, an electrode 37 for measuring the heartbeat of the driver, a microphone (not shown) for performing voice recognition of the driver, and the like.

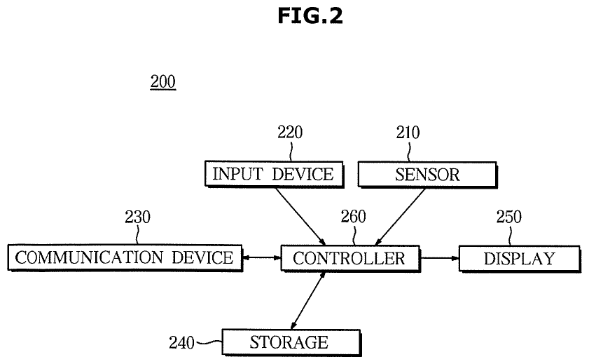

FIG. 2 is a block diagram illustrating some components of an emotion mapping apparatus according to an exemplary embodiment of the present disclosure. An emotion mapping apparatus 200 according to FIG. 2 may be a standalone electronic device as a processor (CPU), or may be a part of the vehicle 100 as an electronic control unit (ECU).

Referring to FIG. 2, the emotion mapping apparatus 200 according to an embodiment may include a sensor 210 for sensing a condition of a user using a plurality of sensors and acquiring information on the condition of the user, an input device 220 for receiving information on the user from the user, a communication device 230 for receiving driving information and traffic information of the vehicle 100 from an external server, a storage 240 for storing various information related to the user and the vehicle 100, a controller 260 for generating an emotion map based on the information received from the sensor 210 and the information stored in the storage 240, a display 250 for displaying an emotion map generated by the controller 260, and the like.

The sensor 210 may sense and measure a user's condition using various sensors provided in the vehicle 100 and transmit the measurement to the controller 260.

The sensor 210 may include various sensors for sensing and acquiring the user's emotion. For example, the sensor 210 may include at least one of a galvanic skin response (GSR) measuring device capable of measuring a condition of the user's skin, a heart rate (HR) meter capable of measuring the user's heart rate, an electroencephalogram (EEG) measuring instrument capable of measuring the user's brain waves, a facial analysis device capable of analyzing the user's facial condition, and an eye tracker capable of tracking the position of the pupils of eyes of the user. The sensors included in the sensor 210 are not limited to those described above, and any other sensors that may measure a person's condition may be included in the sensor 210.

Further, the sensor 210 may sense various information of the vehicle 100 and transmit the result to the controller 260.

The vehicle information may include information about the vehicle itself, internal information of the vehicle, and outside information of the vehicle.

The information about the vehicle itself may include information on a state of the vehicle and whether or not a function of the vehicle is operated. Specifically, the information about the vehicle itself may include various information such as speed, acceleration, and deceleration information of the vehicle 100, activation and pressure information of the accelerator/brake pedal, a seat position, information about an operation state of the heating wire/ventilator, operation information of the air conditioning system, indoor brightness information, indoor fine dust level information, and information about whether the window is opening or closed.

The internal information of the vehicle may be information about what the user or the passenger does inside the vehicle 100. Specifically, the internal information of the vehicle 100 may include information on whether or not the passenger is present, information on the conversation state, information on whether the multimedia is operating, and information on the type of the content played when the multimedia is operated.

The external information of the vehicle 100 may include all external information related to traveling of the vehicle 100. Specifically, the external information of the vehicle 100 may include current time information, position information, traffic situation information of a road and information about a road on which the vehicle 100 is traveling, weather information, and external event information performed on the traveling route of the vehicle 100.

The traffic situation information may include information on whether the current traffic situation is fine or busy, and the road information may include information on traffic lights, crosswalks, road types and forms, and speed limits on the roads.

Such information may be transmitted to the controller 260, and the controller 260 may create an emotion map after determining an emotional condition of the user based on the information, and perform feedback based on the emotional condition and the emotion map of the user.

The input device 220 may receive information on the user and emotion information from the user.

The user information may include body information of the user. For example, the user information may include information about at least one of sex, age, weight, and height of the user, and such information may be input directly from the user.

The emotion of the user may be estimated on the basis of the information obtained from the sensor 210, or in some cases, the user may directly input his/her emotion through the input device 220.

The user may directly input his/her emotion, for example, anger, sadness, boredom, pleasure, etc., through the input device 220. The user may directly input his/her emotion by voice or may input his/her emotion using characters or emoticons.

The communication device 230 may transmit and receive driving information and traffic information of the vehicle 100 with an external server and may receive information on a relationship between a sensor and an emotional factor from the external server.

The driving information of the vehicle 100 may include information on the road on which the vehicle 100 is currently traveling and information on emotions that the other drivers feel on the road on which the vehicle 100 is currently traveling.

The communication device 230 is a hardware device transmitting an analog or digital signal and may communicate with an external server using various methods. The communication device 230 may transmit and receive information with an external server by using various methods such as radio frequency (RF) communication, wireless fidelity (Wi-Fi) communication, Bluetooth communication, Zigbee communication, near field communication (NFC) communication, and ultra-wide band (UWB) communication. However, the communication method is not limited thereto, and any method may be applied as long as it may support communication with an external server.

Although in FIG. 2, the communication device 230 is shown as a single component for transmitting and receiving signals, it is not limited thereto. For example, a transmitter (not shown) for transmitting a signal and a receiver (not shown) for receiving a signal may be separately provided.

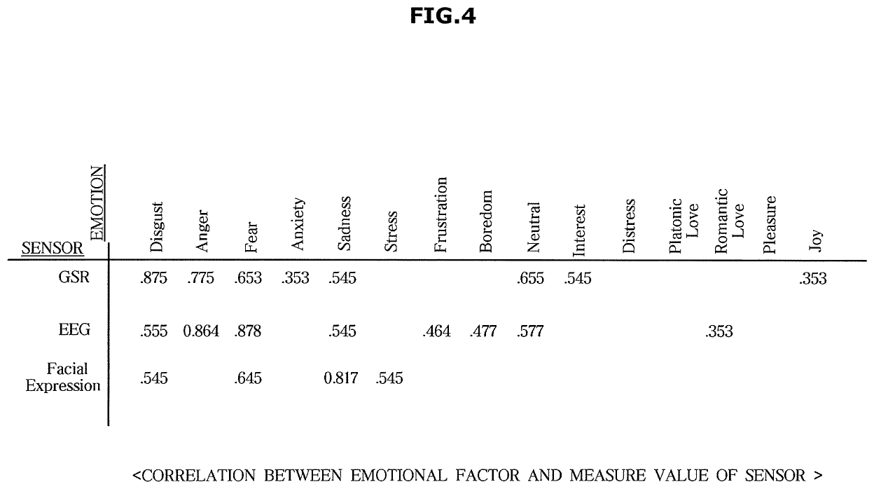

The storage 240 is a computing hardware device and may store various information on the user and the vehicle 100, and information on correlations between sensors and emotional factors. Specifically, as shown in FIG. 4, information on correlations between various sensors and emotional factors may be stored in the storage 240.

The table of FIG. 4, which is an example of relationships between sensors and emotional factors, is a table that classifies correlation information between the GSR measuring device, the EEG measuring instrument, and the facial analysis device and the emotional factors.

Referring to FIG. 4, for the emotional factors of disgust and anger emotional factor, the correlation values with the GSR measuring device are 0.875 and 0.775, respectively, which are considered to have a relatively high relevance with the GSR measuring device. Accordingly, the information measured by the GSR measuring device indicates that the user's emotion is more of disgust or anger than other emotions.

On the other hand, for the emotional factor of joy emotional factor, the correlation value with the GSR measuring device is 0.353, which is considered to have a relatively low relevance with the GSR measuring device. Accordingly, the emotion of joy is less relevant to the information measured by the GSR measuring device than other emotions.

In the case of the EEG measuring instrument, the correlation with the emotional factor of fear is 0.878, which is considered to have a higher relevance than the other emotional factors. Accordingly, it may be determined that the information measured by the EEG measuring instrument has a relatively high relevance with the emotion of fear.

The information shown in the table of FIG. 4 represents results derived from an experiment, and the derived values may be changed according to the experimental environment.

The storage 240 may be implemented with at least one of a nonvolatile memory element such as a read only memory (ROM), a programmable ROM (PROM), an erasable programmable ROM (EPROM), an electrically erasable programmable ROM (EEPROM) and a flash memory, a volatile memory element such as a random access memory (RAM), and a storage medium such as a hard disk drive (HDD) and a CD-ROM for storing various information, but is not limited thereto. The storage 240 may be a memory implemented in a separate chip from a processor, which will be described later in connection with the controller 260, or may be implemented with the processor in a single chip.

The display 250 is an output device for presentation of information in visual or tactile from and may display various information including driving information and a travelling route of the vehicle 100, and may display the emotion map generated by the controller 260. The screen displayed on the display 250 may be controlled by the controller 260.

The display 250 may include a display panel (not shown) for representing the display screen, and the display panel may employ a cathode ray tube (CRT) display panel, a liquid crystal display (LCE) panel, a light emitting diode (LED) panel, an organic light emitting diode (OLED) panel, a plasma display panel (PDP), a field emission display (FED) panel, or the like.

The display 250 may be configured as a touch screen display that receives a touch of the user as an input. In this case, the display 250 may include a display panel (not shown) for displaying an image and a touch panel (not shown) for receiving a touch input. When the display 250 is configured as the touch screen display, the display 250 may perform the function of the input device 220.

The controller 260 may be a processor such as a CPU or more specifically an electronic control unit (ECU), and may control various devices provided in the vehicle 100 and may generate an emotion map based on the information received from the sensor 210 and the information stored in the storage 240.

Specifically, the controller 260 may receive the information on the relationships between the sensors and the emotional factors from the storage 240, extract emotional factors whose relevance with the emotional factors exceeds a preset reference among the values measured by the sensors, acquire information on the emotional state of the user based on the extracted emotional factors, and generate an emotion map in which information on the emotional state of the user acquired is classified according to a preset reference.

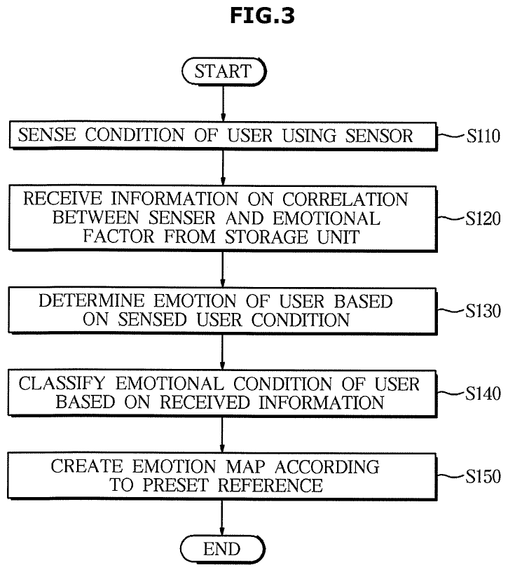

Further, the controller 260 may create an emotion map in a method of classifying information on the emotional state of the user according to preset emotional axes. In addition, the emotional axes may include at least one of positivity, negativity, and excitement. A detailed description thereof will be described with reference to FIGS. 3 to 6.

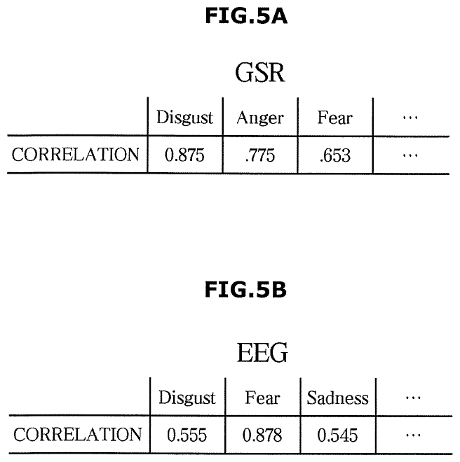

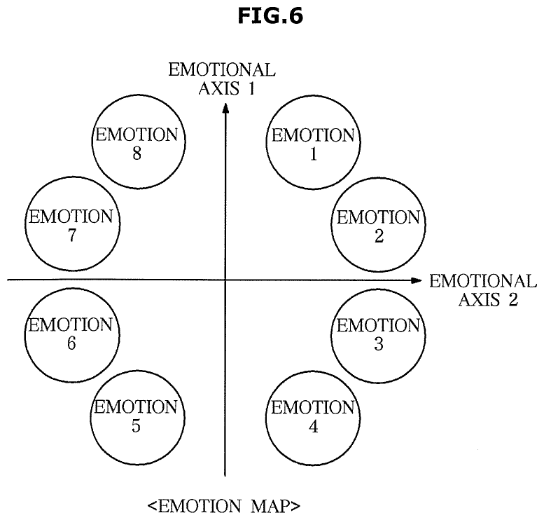

FIG. 3 is a flowchart illustrating a control method of an emotion mapping apparatus according to an embodiment, and FIG. 4 is a table illustrating information on correlations between sensors and emotional factors. FIGS. 5A and 5B are tables illustrating emotional factors extracted as having correlations with sensors exceed a preset reference, and FIG. 6 is a view illustrating an emotion map generated according to an embodiment.

Referring to FIG. 3, an emotion mapping apparatus 200 may sense a condition of a user using various sensors (S110).

As described with reference to FIG. 2, the sensors may include at least one of a GSR measuring device capable of measuring a condition of the user's skin, an HR meter capable of measuring the user's heart rate, an EEG measuring instrument capable of measuring the user's brain waves, a facial analysis device capable of analyzing the user's facial state, and an eye tracker capable of tracking the position of a pupil of an eye of the user.

After sensing the condition of the user, the emotion mapping apparatus 200 may receive information on the correlations between the sensors and the emotional factors stored in the storage 240 (S120).

Specifically, the information on the correlations between the emotional factors and the sensor measurement values as shown in the table of FIG. 4 may be received from the storage 240 or the external server. The information on the correlations between the sensors and the emotional factors has been described above, and thus, the description thereof will not be repeated.

After the information on the user's condition is sensed and the information on the correlations between the sensors and the emotional factors is received, the emotion mapping apparatus 200 may determine an emotion of the user based on the information (S130).

Describing the process of S130 with reference to FIGS. 4 and 5, if there is a sensor used for measurement among the sensors shown in the table of FIG. 4, the emotion mapping apparatus 200 may extract information on the relationship between the sensor used for the measurement and the emotional factor associated with the sensor. In addition, the emotion mapping apparatus 200 may extract not the information on all the emotional factors, but the information on the emotional factors whose relevance exceeds a preset reference.

For example, as shown in FIGS. 5A and 5B, if the user's condition is sensed using the GSR measuring device and the EEG measuring instrument among several sensors, the information on the emotional factors related to the GSR measuring device and the EEG measuring instrument may be extracted, in which case the information on the emotional factors whose relevance exceeds the preset reference may be extracted.

As shown in FIGS. 5A and 5B, emotions of disgust, anger and fear are highly related to the GSR measuring device, and thus extracted as the emotional factors having high relevance. In the case of the EEG measuring instrument, emotions of disgust, fear and sadness are extracted as the emotional factors having high relevance.

Although FIGS. 5A and 5B show the emotional factors having a correlation value of 0.5 or more when the present reference corresponds to 0.5 the preset reference is not limited thereto but may be variously set according to the environment around the user or set by the user.

The controller 260 may extract the emotional factors having high relevance, and then infer the emotional condition of the user based on the extracted emotional factors. For example, referring to FIGS. 5A and 5B, since it was determined that the GSR measuring device, the EEG measuring instrument, and two sensors have high relevance to emotions of disgust and anger, the emotion mapping apparatus 200 may determine that the user is currently in the same or similar emotional condition as the emotions.

When the emotional condition of the user is determined, the emotion mapping apparatus 200 may classify the emotional condition of the user based on the determination (S140), and create an emotion map according to the preset reference (S150).

FIG. 6 shows an emotion map in which various emotional conditions of a user are classified based on preset emotional axes, and the emotional condition of the user may be expressed at various positions. The emotional axes may be set based on the emotions measurable by the sensors.

For example, emotional axis 1 may be positivity that may be measured by analysis of the user's voice or face, and emotional axis 2 may be excitement or activity that may be measured by the GSR measuring device or the EEG measuring instrument.

Accordingly, if it is measured in the process of S130 that the user's emotional condition is in a state of high positivity and high excitement, the emotional axis 1 may be used as a positivity axis on the emotion map, the emotion axis 2 may be used as an excitement axis, and the user's current emotional condition may be located at emotion 1 or emotion 2. On the other hand, if it is measured that the user's emotional condition is in a state of high negativity and high excitement, the user's current emotional condition may be located at emotion 3 or emotion 4.

The positivity and excitement, which may be the reference of the emotional axis, are only an example, and any other emotions that may be measured by the sensors may be the reference of the emotional axis.

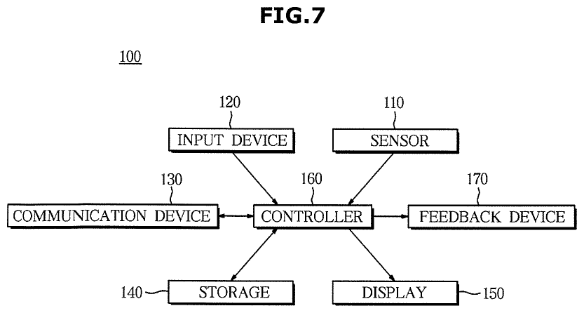

FIG. 7 is a block diagram illustrating some components of a vehicle according to an exemplary embodiment of the present disclosure.

Referring to FIG. 7, the vehicle 100 according to an embodiment may include a sensor 110 for sensing a condition of the user using sensors and acquiring information on the condition of the user, an input device 120 for receiving information on the user from the user, a communication device 130 for receiving driving information and traffic information of the vehicle 100 from an external server, a storage 140 for storing various information related to the user and the vehicle 100, a display 150 for displaying an emotion map generated, a controller 160 for generating the emotion map based on the information received from the sensor 110 and the information stored in the storage 140 and for controlling a feedback device 170 to control the current emotional condition of the user to a target emotion, and the feedback device 170 including various devices provided in the vehicle 100.

The sensor 110, the input device 120, the communication device 130, the storage 140, the display 150, and the controller 160 as shown in FIG. 7 are basically the same as the sensor 210, the input device 220, the communication device 230, the storage 240, the display 250, and the controller 260 as shown in FIG. 2, respectively, and thus overlapping descriptions will not be repeated, but the storage 140, the controller 160, and the feedback device 170, which have additional features, will be focused.

The storage 140 may store various information related to the user and the vehicle 100, information on correlations between the sensors and the emotional factors, and information on correlations between the emotional factors and the feedback elements.

Since the information on the correlations between the sensors and the emotional factors was described with reference to FIG. 4, further explanation will be omitted and the information about the correlations between the emotional factors and the feedback elements will now be described.

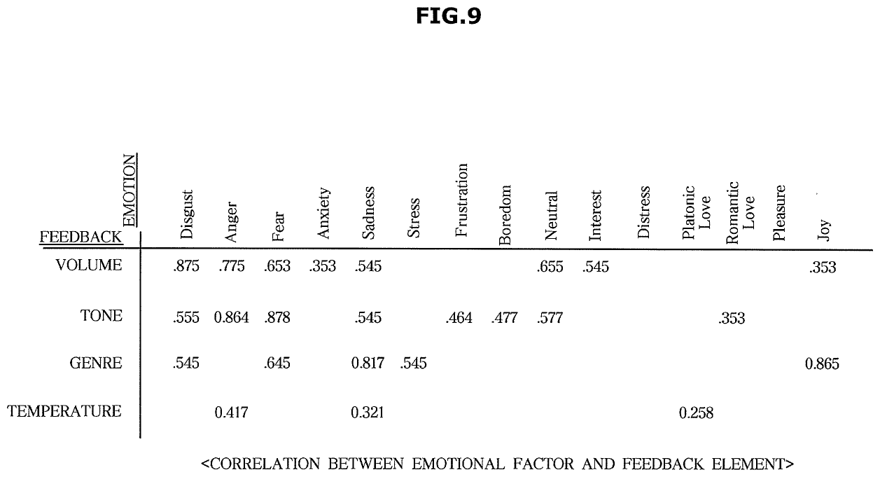

FIG. 9 shows an example of a table that classifies information on correlations between a plurality of emotions and feedback elements (volume, tone, genre, temperature).

Referring to FIG. 9, it may be seen that the emotion of anger is correlated with volume, tone and temperature, and that the correlation with the tone is 0.864, which is the highest. Accordingly, when the user's emotional condition is determined to be anger, it may be seen that changing the emotional condition of the user by regulating the tone is the most efficient feedback method.

In another example, it may be seen that the emotion of sadness is correlated with volume, tone, genre and temperature, and that the correlation with the genre is 0.817, which is the highest. Accordingly, when the user's emotional condition is determined to be sadness, it may be seen that changing the emotional condition of the user by regulating the genre is the most efficient feedback method.

Further, it may be seen that the emotion of joy is correlated with volume and genre, and that the correlation with the genre is 0.865, which is the highest. Accordingly, when the user's emotional condition is determined to be joy, it may be seen that keeping the user joyous by regulating the genre is the most efficient feedback method.

The information represented in the table of FIG. 9 shows measurements from an experiment, and the values derived from the experiment may be changed according to environments of the experiment.

The controller 160 may control various devices provided in the vehicle 100, and generate an emotion map based on information received from the sensor 110 and the information stored in the storage 140.

Specifically, the controller 160 may fetch information about relationships between the sensors and the emotional factors from the storage 140, extract emotional factors having relevance that exceeds a preset reference among the values measured by the sensors, acquire information on the emotional condition of the user based on the extracted emotional factors, and generate an emotion map in which information on the emotional condition of the user acquired is classified according to a preset reference.

Further, the controller 160 may fetch information about relationships between the sensors and the emotional factors and feedback information necessary for the user in relation to the emotional factors from the storage 140, acquire information on the current emotional condition of the user based on the values measured by the sensors, and control the feedback device 170 provided in the vehicle 100 so that the current emotional condition of the user reaches a target emotion.

Specifically, the controller 160 may control the feedback device 170 so that the emotional condition of the user is maintained at a first emotion when the current emotional condition of the user corresponds to the first emotion, and may control the feedback device 170 so that the emotional condition of the user reaches the first emotion when the current emotional condition of the user corresponds to a second emotion.

The first emotion and the second emotion indicate opposite emotions. For example, the first emotion may indicate pleasure or happiness including many positive emotional factors, and the second emotion may indicate sadness or anger including many negative emotional factors. In the emotion map of FIG. 6, emotion 1, emotion 2, emotion 7, and emotion 8 may belong to the first emotion, and emotion 3, emotion 4, emotion 5, and emotion 6 may belong to the second emotion.

If the current emotional condition of the user corresponds to the second emotion having many negative emotional factors, the controller 160 may control the feedback device 170 so that the user's emotional condition reaches the first emotion having many positive emotional factors.

The first emotion and the second emotion are not limited to the emotion having many positive emotional factors or the emotion having many negative emotional factors but may be classified into various references according to the setting of the user.

The feedback device 170 includes a hardware device and may include at least one of a multimedia device, an air conditioner, a display, a speaker, and a ventilator, and the controller 160 may control the user's emotional condition to reach a target emotion by controlling at least one of the volume, genre, equalizer, tone, and acoustic wave band of the music played in the vehicle 100.

In FIG. 9, although the feedback element for changing the emotional state of the user is described as music related elements, the feedback element is not necessarily limited to music related elements.

For example, if the user's emotion is ‘afraid’ and/or ‘surprised’, the feedback element correlates with ‘afraid’ and/or ‘surprised’ may include tightening speed of the seat belt, tightening strength of the seat belt, operating sensitivity of the steering wheel. If the feedback element having the highest correlation with the afraid emotion is the tightening strength of the seat belt, it is possible to change the afraid emotion of the user to the comfortable emotion by adjusting the tightening strength of the seat belt.

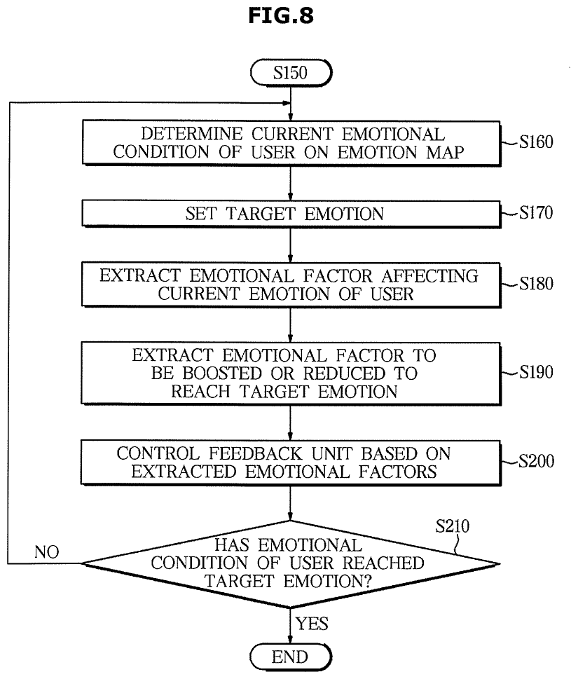

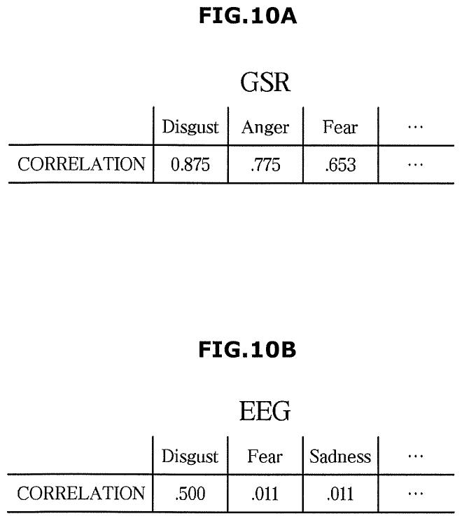

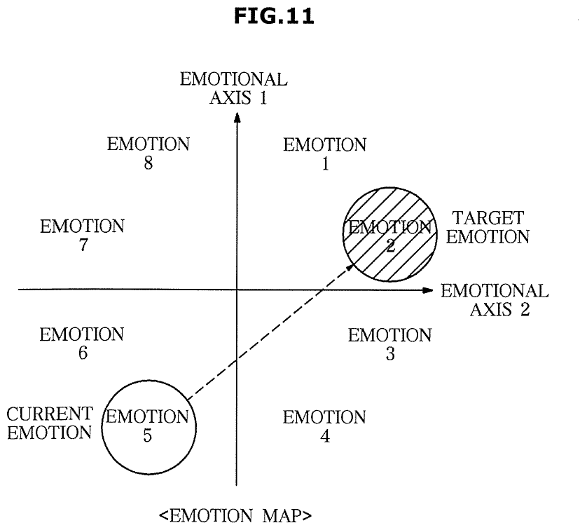

FIG. 8 is a flowchart illustrating a control method of a vehicle according to an embodiment, FIG. 9 is a table representing information about correlations between emotional factors and feedback elements, and FIGS. 10A and 10B are tables representing emotional factors extracted as having correlations with the feedback elements exceeding a preset reference. FIGS. 11 to 13 are diagrams illustrating a method of making the user's emotional condition reach a target emotion.

In the flowchart of FIG. 8, the starting point is shown as the step of S150 of FIG. 3. However, the step of S160 is not always executed after the step of S150 but may be executed independently of the steps of S110 to S150.

Referring to FIG. 8, the controller 160 may determine which position on the emotion map the current emotional condition of the user is (S160).

For example, the controller 160 may determine whether the current emotion of the user is located at emotion 1 or emotion 5 on the emotion map shown in FIG. 6.

If a position where the user's current emotional condition is located on the emotion map is determined, the controller 160 may set the target emotion of the user (S170).

As shown in FIG. 11, if it is determined that the current emotional condition of the user is located at emotion 5, the target emotion may be determined so that the emotional condition of the user reaches emotion 2. The target emotion shown in FIG. 11 is merely an example, and may be set to be at other various positions.

For example, if the current emotional condition of the user is highly negative, the target emotion may be set to a direction of increasing the positivity or increasing the excitement. In addition, if the user's emotional condition is sensed as being highly positive, the target emotion may be set to maintain the current emotional condition.

The target emotion is not fixed but may be changed to any of various target emotions according to the user's environment. This target emotion may be preset by the user. For example, if the user always wants to be pleased, the target emotion may be set to being pleased, and if the user wants to be melancholy, the target emotional condition may be set to being melancholy.

If the target emotional condition is set, the controller 160 may extract emotional factors that affect the current emotion of the user (S180), and may extract emotional factors to be boosted or reduced to reach the target emotion from among the extracted emotional factors (S190).

Specifically, after the controller 160 may analyze emotional factors affecting the user's emotional condition, classify the emotional factors into a first group to which the positive emotional factors belong and a second group to which the negative emotional factors belong, and control the feedback device 170 to increase the emotional factors belonging to the first group and decrease the emotional factors belonging to the second group.

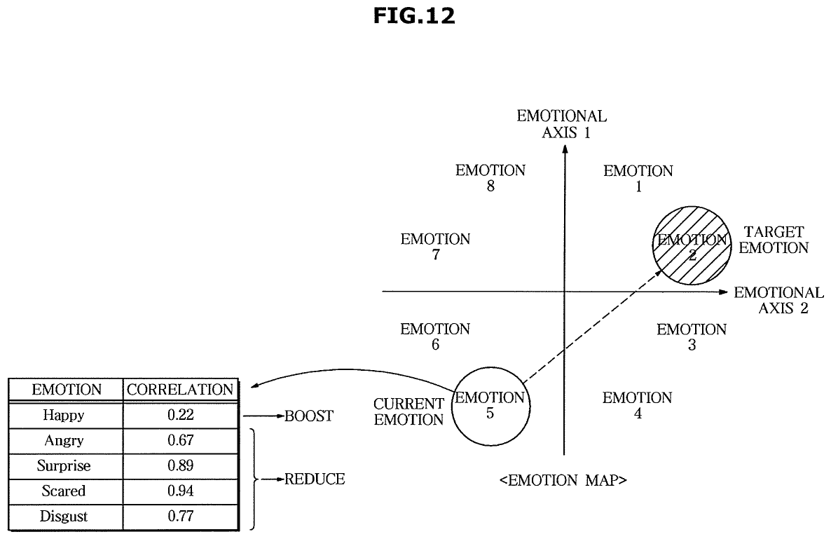

For example, as shown in FIG. 12, if it is determined that the current emotional condition of the user is located at emotion 5 on the emotion map, the emotional factors affecting the current emotional condition may be extracted.

In FIG. 12, it may be seen that the emotional factors affecting the user's current emotion are happiness, anger, surprise, scare, and disgust. The happiness may be classified into the first group to which the positive emotional factors belong, and anger, surprise, scare, and disgust may be classified into the second group to which the negative emotional factors belong.

The controller 160 may boost or reduce the extracted emotional factors based on the set target emotion. For example, if the set target emotion is pleasure, the emotional factors of the first group to which the positive emotional factors belong may be boosted, and the emotional factors of the second group to which the negative emotional factors belong may be reduced. Conversely, if the set target emotion is melancholic emotion, the emotional factors of the first group may be reduced and the emotional factors of the second group may be boosted.

If the emotional factors to be boosted or reduced are extracted, the controller 160 may control the feedback device 170 based on the extracted emotional factors (S200).

Referring to FIG. 12, when the set target emotion is pleasure, the controller 160 may control the feedback device 170 so that the correlation of happiness increase since the correlation of happiness corresponding to the positive emotional factor in the current emotional condition of the user is low, and the correlations of anger, surprise, and disgust are reduced since the correlations of the emotions corresponding to the negative emotional factors are high. Here, the correlation indicates an extent to which each emotional factor affects the current emotional condition of the user.

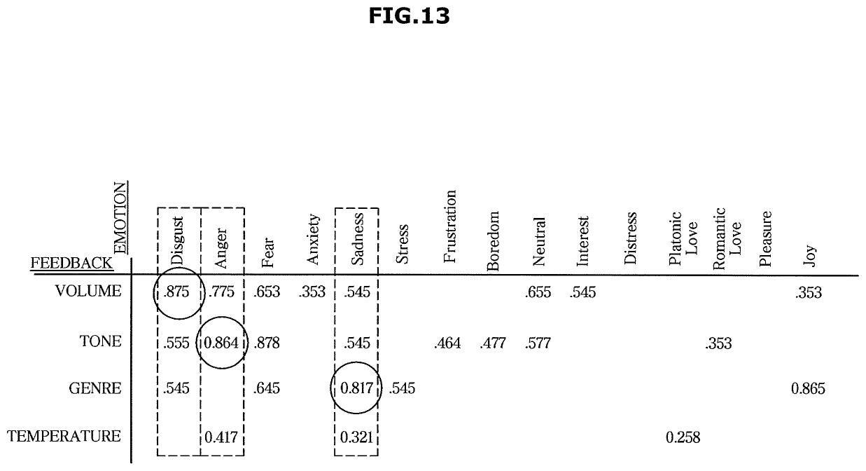

Referring to FIG. 13, if the emotional factor affecting the emotional condition of the user is disgust, it may be seen to have the highest correlation with the volume. Accordingly, the degree to which the emotion of disgust affects the user's emotional condition may be reduced by adjusting the volume.

For the emotion of anger, the tone is the most highly correlated feedback element, and thus the influence of the emotional factor of anger on the emotional condition of the user may be reduced by adjusting the tone. In addition, for the emotion of sadness, the genre is the most highly correlated feedback element, and thus the influence of the emotional factor of sadness on the emotional condition of the user may be reduced by adjusting the genre.

Consequently, the vehicle 100 may change the mood of the user by controlling the feedback elements having high correlations with the emotional factors to be boosted or reduced.

As is apparent from the above, the vehicle 100 and the control method of the vehicle 100 according to an embodiment may provide the user with appropriate feedback based on the mood of the user determined in real time, leading to the benefit of providing the user with a vehicle driving environment to his/her liking.

Although the present disclosure has been described in connection with certain exemplary embodiments and drawings, various modifications and changes may be made by those skilled in the art without departing from the scope of the invention. For example, appropriate results could be achieved even though the described techniques are performed in a different order than the described method, and/or the components of the described systems, structures, devices, circuits, and the like are combined in different ways from the described methods, or replaced by other components or equivalents. Therefore, it is apparent that other embodiments and equivalents to the claims are within the scope of the following claims.

What is claimed is:

1. A vehicle comprising:
a sensor configured to sense a condition of a user using at least one sensor;
a storage configured to store information on a relationship between the at least one sensor and an emotional factor and feedback information for the user with regard to the emotional factor; and
a controller configured to acquire information on a current emotional condition of the user based on values measured by the at least one sensor and to control a feedback device of the vehicle so that the current emotional condition of the user reaches a target emotion.

2. The vehicle according to claim 1, wherein the controller is configured to classify the current emotional condition of the user and the target emotion according to a reference, and then to control the feedback device based on a classification result.

3. The vehicle according to claim 1, wherein the controller is configured to, when the current emotional condition of the user corresponds to a first emotion, control the feedback device so that the emotional condition of the user is maintained at the first emotion.

4. The vehicle according to claim 1, wherein the controller is configured to, when the current emotional condition of the user corresponds to a second emotion, control the feedback device so that the emotional condition of the user reaches a first emotion.

5. The vehicle according to claim 1, wherein the controller is configured to extract emotional factors affecting the current emotional condition of the user, and then control the feedback device to raise or reduce the extracted emotional factors.

6. The vehicle according to claim 5, wherein the controller is configured to, when the emotional factors belong to a first group, control the feedback device to raise the emotional factors.

7. The vehicle according to claim 5, wherein the controller is configured to, when the emotional factors belong to a second group, control the feedback device to reduce the emotional factors.

8. The vehicle according to claim 1, wherein the feedback device comprises at least one of a multimedia device, an air conditioner, a display, a speaker, or a ventilator disposed in the vehicle.

9. The vehicle according to claim 8, wherein the controller is configured to control at least one of volume, genre, equalizer, tone, or acoustic wave band of music played in the vehicle.

10. The vehicle according to claim 1, further comprising:
an input device configured to receive information on the target emotion from the user.

11. A control method of a vehicle comprising steps of:
sensing a condition of a user using at least one sensor;
receiving, by a controller, information on a relationship between the at least one sensor and an emotional factor and feedback information for the user with regard to the emotional factor stored in a storage; and
acquiring, by a controller, information on the current emotional condition of the user based on values measured by the at least one sensor and controlling a feedback device of the vehicle so that the current emotional condition of the user reaches a target emotion.

12. The control method according to claim 11, wherein the step of controlling the feedback device comprises classifying the current emotional condition of the user and the target emotion according to a reference, and controlling the feedback device based on a classification result.

13. The control method according to claim 11, wherein the step of controlling the feedback device comprises, when the current emotional condition of the user corresponds to a first emotion, controlling the feedback device so that the emotional condition of the user is maintained at the first emotion.

14. The control method according to claim 11, wherein the step of controlling the feedback device comprises, when the current emotional condition of the user corresponds to a second emotion, controlling the feedback device so that the emotional condition of the user reaches a first emotion.

15. The control method according to claim 11, wherein the step of controlling the feedback device comprises extracting emotional factors affecting the current emotional condition of the user, and controlling the feedback device to raise or reduce the extracted emotional factors.

16. The control method according to claim 15, wherein the step of controlling the feedback device comprises, when the emotional factors belong to a first group, controlling the feedback device to raise the emotional factors.

17. The control method according to claim 15, wherein the step of controlling the feedback device comprises, when the emotional factors belong to a second group, controlling the feedback device to reduce the emotional factors.

18. The control method according to claim 11, wherein the feedback device includes at least one of a multimedia device, an air conditioner, a display, a speaker, or a ventilator disposed in the vehicle.

19. The control method according to claim 18, wherein the step of controlling the feedback device comprises controlling at least one of volume, genre, equalizer, tone, or acoustic wave band of music played in the vehicle.

20. The control method according to claim 11, further comprising:
receiving information on the target emotion from the user.
